(19) United States
(12) Patent Application Publication
(10) Pub. No.: US 2019/0001001 A1
(43) Pub. Date: Jan. 3, 2019

(54) DRUG DESIGN FOR APPLICATION-DEPENDENT PAYLOAD, CONTROLLED PHARMACOKINETIC DISTRIBUTION, AND RENAL CLEARANCE

(71) Applicants: General Electric Company, Schenectady, NY (US); University of California San Francisco, San Francisco, CA (US)

(72) Inventors: Paul Francis Fitzgerald, Schenectady, NY (US); Michael Ernest Marino, Niskayuna, NY (US); Peter Michael Edic, Albany, NY (US); Daniel Eugene Meyer, Rexford, NY (US); Peter J. Bonitatibus, Jr., Saratoga Springs, NY (US); Robert Edgar Colborn, Schenectady, NY (US); Benjamin Ming-Yueh Yeh, Hillsborough, CA (US)

(21) Appl. No.: 15/640,880

(22) Filed: Jul. 3, 2017

(51) Int. Cl. A61K 49/04

(52) U.S. Cl. CPC A61K 49/0423

(57) ABSTRACT

Design and use of an administered drug in the form of a nanoparticle or molecule is described. In certain examples, the nanoparticle has a core and a shell surrounding the core. The core may be configured or designed to provide useful X-ray attenuating properties, gamma ray emission properties, magnetic properties, or therapeutic effects. In certain aspects, the nanoparticle or molecule is sized so as to either distribute from or remain in the blood pool, while still being eliminated by the kidneys.

### STATEMENT REGARDING FEDERALLY SPONSORED RESEARCH & DEVELOPMENT

This invention was made with Government support under contract number R01 EB015476 awarded by National Institutes of Health. The Government has certain rights in the invention.

### BACKGROUND

Non-invasive imaging technologies allow images of the internal structures or features of a patient to be obtained. In particular, such non-invasive imaging technologies rely on various physical principles, such as the differential transmission of X-ray photons through the target volume or the reflection of acoustic waves, to acquire data and to construct images or otherwise represent the internal features of the subject.

For example, in X-ray-based imaging technologies, X-ray radiation spans a subject of interest, such as a human patient, and a portion of the radiation impacts a detector where the intensity data is collected. In digital X-ray systems, a detector produces signals representative of the amount or intensity of radiation impacting discrete pixel regions of a detector surface. The signals may then be processed to generate an image that may be displayed for review.

In one such X-ray-based technique, known as computed tomography (CT), a scanner may project X-ray beams from an X-ray source at numerous view angle positions about a patient. The X-ray beams are attenuated as they traverse the object and are detected by a set of detector elements which produce signals representing the intensity of the incident X-ray intensity on the detector. The signals are processed to produce data representing the line integrals of the linear attenuation coefficients of the object along the X-ray paths. These signals are typically called “projection data” or just “projections”. By using reconstruction techniques, such as filtered backprojection, images may be generated that represent a volume or a volumetric rendering of a region of interest of the patient or imaged object. In a medical context, pathologies or other structures of interest may then be located or identified from the reconstructed images or rendered volume.

To enhance the image contrast between certain types of anatomy of interest and other tissues, a contrast agent may be employed that, when administered, increases the opacity of the tissues in which it is present. For example, in clinical X-ray/CT imaging, the anatomy of interest may be vasculature or organ parenchyma that contains blood, which is otherwise difficult to distinguish from adjoining tissue at X-ray in the absence of a contrast agent.

However, current imaging contrast agents have various limitations. For example, the relatively small size of iodinated small molecules allows them to almost immediately begin to distribute from the blood pool into the interstitial fluid immediately, substantially diluting the contrast agent in the minutes following administration. This limits the available time in which acquired images contain the maximum contrast agent concentration in blood vessels and organs of interest. Therefore, even within the acquisition window, the distribution effect may impact the comparability of images obtained at different times in the acquisition window due to the contrast enhancement being at least partly dependent on the concentration of the agent within the anatomic compartments of interest in the imaged volume. Further, there is an upper limit on the size of the molecules that comprise such agents, as larger molecules may not be removed efficiently by the patient's kidneys. Removal by the kidneys is important so that the agent is not retained within the patient's body in such organs as kidney, liver and spleen. Rapid renal clearance generally reduces the potential for toxicity by minimizing tissue exposure to the agent.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible embodiments. Indeed, the invention may encompass a variety of forms that may be similar to or different from the embodiments set forth below.

In one aspect, an agent is provided that can be injected into a subject (e.g. patient). In accordance with this aspect, the agent comprises nanoparticles or molecules sized to effect a specific degree of distribution or lack of distribution between tissues, organs, or bodily compartments of the subject while still being eliminated by the kidneys

In a further aspect, a method is provided for performing a contrast-enhanced image acquisition. In accordance with this aspect, a size of a patient or an anatomical region within the patient to be imaged is determined. Based on the size of the patient or anatomical region, an X-ray energy spectrum to be used to acquire one or more images of the patient or anatomical region within the patient is determined. Based on one or both of the anatomical size or the X-ray energy spectrum, one or more X-ray attenuating elements are selected to be used as a constituent of a contrast agent. The contrast agent is administered to the patient. The contrast agent comprises nanoparticles or molecules having a size selected so as to effect a specific degree of distribution or lack of distribution between tissues, organs, or bodily compartments of the patient while still being eliminated by the kidneys. One or more contrast-enhanced images of the patient are acquired.

In an additional aspect, a method is provided for performing a procedure using one or more types of drugs that can be injected into a patient. In accordance with this aspect the one or more types of drugs are administered to a patient as part of a procedure. The drugs, when more than one is present, may be injected simultaneously or sequentially. One or more of the types of drugs comprise nanoparticles or molecules having a size selected so as to effect a specific degree of distribution or lack of distribution between tissues, organs, or bodily compartments of the patient while still being eliminated by the kidneys.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a schematic illustration of an embodiment of a computed tomography (CT) system configured to acquire CT images of a patient and process the images in accordance with aspects of the present disclosure;

FIG. 2 depicts a curve illustrating the permeability of endothelial monolayer to molecules of different Stokes-Einstein radii;

FIG. 3 depicts the concentration of the contrast agent iopromide in pig plasma, illustrated as a function of time;

FIG. 4 depicts CT image contrast for various elements over a range of peak X-ray energies;

FIG. 5 depicts a cutaway and chemical view of an example of a contrast agent nanoparticle, in accordance with aspects of the present approach;

FIG. 6 depicts CT images of pigs encased within adipose-equivalent encasements after injection of the pig with a TaCZ nanoparticle contrast agent or iopromide, a conventional iodinated small-molecule contrast agent;

FIG. 7 depicts results of a multi-reader assessment of CT images of pigs generated using a TaCZ nanoparticle contrast agent or iopromide, a conventional iodinated small-molecule contrast agent;

FIG. 8 depicts study results assessing TaCZ nanoparticles or iopromide in pig plasma; and

FIG. 9 depicts study results assessing TaCZ nanoparticles or iopromide in pig urine.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While the following discussion is generally provided in the context of medical imaging, it should be appreciated that the present techniques are not limited to such imaging contexts. Indeed, the provision of examples and explanations in such an imaging context is only to facilitate explanation by providing instances of real-world implementations and applications. However, the present approaches may also be utilized in other drug or pharmacological agent delivery contexts including, but not limited to, delivery of cancer treatment drugs, PET tracers (molecules emitting gamma radiation), magnetic elements, and/or multiple or mixed payloads of differing contrast agents and or contrast agents combined with therapeutics. In general, the present approaches may be desirable in any agent delivery context where controlled pharmacokinetic distribution and/or renal clearance are factors.

As discussed in greater detail herein, one type of administered agent that may benefit from the present approach are contrast agents, which are used in medical imaging to enhance the image contrast between the anatomy of interest and other tissues. For instance, in clinical X-ray or computed tomography (CT) imaging, the anatomy of interest may be vasculature or organ parenchyma that contains blood, in which case the contrast agent is injected into the bloodstream where it increases the relative opacity of the volume in which it is present.

The efficacy of the contrast agent depends on various factors, including the X-ray attenuating element in the contrast agent, the injected concentration of that element, the diameter of the patient/anatomy being scanned and the associated X-ray spectrum that is used, the pharmacokinetic (PK) properties of the contrast agent, the hemodynamic physiology of the organ(s) and tissue(s) being scanned, and the time after contrast agent injection at which the scan is performed. As discussed herein, the size of the molecule or nanoparticle that comprises the contrast agent may have consequences in terms of blood pool distribution (or more generally, pharmacokinetic distribution) and renal clearance. The present approach addresses certain of these issues in the context of not only X-ray based contrast agents, but also contrast agents for use in other modalities, which may be subject to similar issues, as well as more generally to any administered drug where one or both of controlled pharmacokinetic distribution and renal clearance are of interest. Herein, the distribution of interest is between tissues, organs, or bodily compartments. Furthermore, the present approach addresses the administration of multiple contrast agents and/or drugs, administered either simultaneously or sequentially, where the pharmacokinetic properties and/or image contrast-enhancing properties of each drug are designed for best efficacy when administered in combination with the other drug(s).

As will be appreciated, the sizes of the nanoparticles used in the various embodiments discussed below are a focus of the present disclosure. Nanoparticles and molecules can take various shapes and forms, including spheres, ellipses, rods, and so forth. In the following discussion, the relevant size of the molecules or nanoparticles might be the largest dimension, the smallest dimension, the hydrodynamic diameter, the hydrodynamic radius, the Stokes radius, or some other estimate of the size, depending on the biological structure with which the molecule or nanoparticle interacts. In the context of molecules and nanoparticle, the term “size”, as used below, is meant to convey the relevant size to produce the observed biological effect or to achieve the desired biological effect; use of the term “size” does not imply a limitation in shape or form, or the size in a particular dimension. Furthermore, conventional, small-molecule contrast agents are typically monodisperse in size, i.e. all the molecules are identical in size; however, nanoparticle formulations are generally polydisperse in size, i.e., nanoparticle formulations will generally have a distribution of sizes. The size distribution may be a Gaussian distribution, but is not necessarily so. Herein, the “nominal nanoparticle size” refers to the mode of the size distribution; the “size range minimum” refers to the size greater than which a large majority (e.g. approximately 90-95%) of the nanoparticles is included; the “size range maximum” refers to the size less than which a large majority (e.g. approximately 90-95%) of the nanoparticles is included; and the “size range” refers to all sizes between the size range minimum and the size range maximum.

To facilitate explanation, certain examples are discussed herein that explain the present approach as it may relate to the delivery of contrast agents in the context of medical imaging systems. As a specific example, a brief explanation of the principles of operation of one such system (here a CT system) that may be used to generate contrast-enhanced images will be initially provided so that the context in which a contrast agent may be employed is more apparent. As may be appreciated however, this example is intended only to provide a framework and background to better understand certain aspects of delivery of an agent, such as a contrast agent, in a medically-useful context, and should not be viewed as limiting the present approaches to either contrast agents or to contrast agents for use in CT imaging. Indeed, the present approach may be beneficial in various situations where a controlled pharmacokinetic distribution and/or renal clearance are issues. Further, even in the image contrast context, the present approach may be useful for the delivery of contrast for various imaging modalities in addition to CT, including, but not limited to, magnetic resonance imaging (MM) and positron emission tomography (PET).

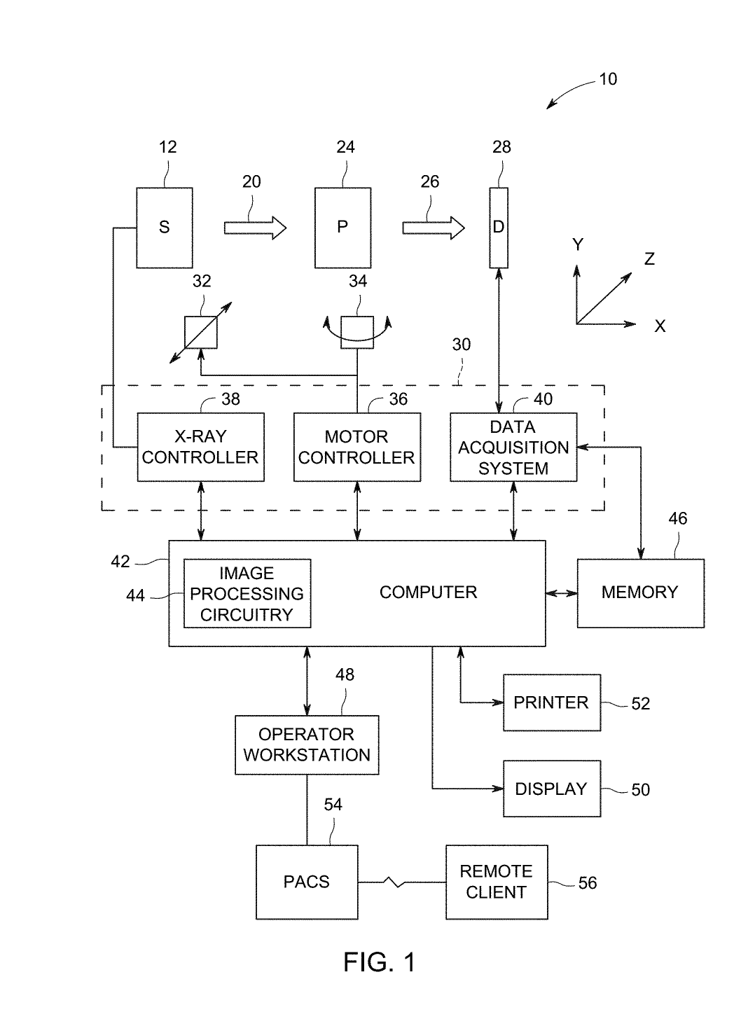

In this context, FIG. 1 illustrates an embodiment of a CT imaging system 10 for acquiring and processing image data, including image data of volumes in which contrast agent may be present. In particular, the computed tomography system 10 acquires X-ray projection data and reconstructs the projection data into volumetric reconstructions for display and analysis. To image certain substances and structures that are otherwise relatively indistinguishable from surrounding tissue at X-ray, a contrast agent is administered to the patient that increases X-ray opacity in areas in which the contrast agent is present, such as the blood vessels or other vasculature as well as organ parenchyma.

With this in mind, the CT imaging system 10 includes one or more X-ray sources 12 which generate X-ray photons during an imaging session. The generated X-ray beam 20 passes into a region in which the subject (e.g., a patient 24) is positioned. The subject attenuates at least a portion of the X-ray photons in the beam 20, resulting in attenuated X-ray photons 26 that impinge upon a detector array 28 formed by a plurality of detector elements (e.g., pixels) as discussed herein. As pertaining to the present discussion, some portion of the X-ray attenuation may be attributed to one or multiple contrast agent(s) administered to the patient prior to and/or during imaging so as to be present at the time of imaging in the region of interest.

The detector 28 typically defines an array of detector elements, each of which produces an electrical signal when exposed to X-ray photons. The electrical signals are acquired and processed to generate one or more projection datasets. In the depicted example, the detector 28 is coupled to the system controller 30, which commands acquisition of the digital signals generated by the detector 28.

A system controller 30 commands operation of the imaging system 10 and may process the acquired data. The system controller 30 may furnish power, focal spot location, control signals and so forth to the X-ray source 12 (such as via the depicted X-ray controller 38) and may control operation of the CT gantry (or other structural support to which the X-ray source 12 and detector 28 are attached), and/or the translation and/or inclination of the patient support over the course of an examination.

In addition, the system controller 30, via a motor controller 36, may control operation of a linear positioning subsystem 32 and/or a rotational subsystem 34 used to move the subject 24 and/or components of the imaging system 10, respectively. Such components facilitate the acquisition of projection data at different positions and angles with respect to the patient, which in turn allows volumetric reconstruction of the imaged region.

The system controller 30 may include a data acquisition system (DAS) 40. The DAS 40 receives data collected by readout electronics of the detector 28, such as digital signals from the detector 28. The DAS 40 may then convert and/or process the data for subsequent processing by a processor-based system, such as a computer 42. The computer 42 may include or communicate with one or more non-transitory memory devices 46 that can store data processed by the computer 42, data to be processed by the computer 42, or instructions to be executed by image processing circuitry 44 of the computer 42.

The computer 42 may also be adapted to control features enabled by the system controller 30 (i.e., scanning operations and data acquisition), such as in response to commands and scanning parameters provided by an operator via an operator workstation 48. The system 10 may also include a display 50 coupled to the operator workstation 48 that allows the operator to view relevant system data, imaging parameters, raw imaging data, reconstructed images or volumes, and so forth. Additionally, the system 10 may include a printer 52 coupled to the operator workstation 48 and configured to print any desired measurement results. The display 50 and the printer 52 may also be connected to the computer 42 directly (as shown in FIG. 1) or via the operator workstation 48. Further, the operator workstation 48 may include or be coupled to a picture archiving and communications system (PACS) 54. PACS 54 may be coupled to a remote system or client 56, radiology department information system (RIS), hospital information system (HIS) or to an internal or external network, so that others at different locations can gain access to the image data.

With the preceding discussion of an overall imaging system 10 in mind, it may be appreciated that the CT imaging system 10 is one type of imaging system that, for certain imaging procedures, may benefit from the use of contrast agents designed and administered in accordance with the present approach. In particular, such agents may have improved characteristics for imaging by such as system, as discussed herein.

With respect to contrast agents that may be employed to acquire images, such as vascular images, using a CT system 10 as shown in FIG. 1, current clinical injectable CT/X-ray contrast agents are typically iodinated small molecules (i.e., the attenuation element is fixed at iodine) with the molecule sizes on the order of approximately 1 nm to 2 nm, resulting in their having nearly identical pharmacokinetic (PK) properties (e.g., distribution rate constant (α), distribution half-life (T½ₐ), clearance rate constant (β), clearance half-life (T½), and so forth) that may not be ideal. For the present purpose, it may be understood that for contrast agents at a suitable clinical concentration (i.e., 240-400 mg/mL), viscosity of up to ˜20 mPa·s and osmolality up to ˜1600 mOsm are acceptable, though osmolality of ˜280 mOsm is preferred for patient comfort.

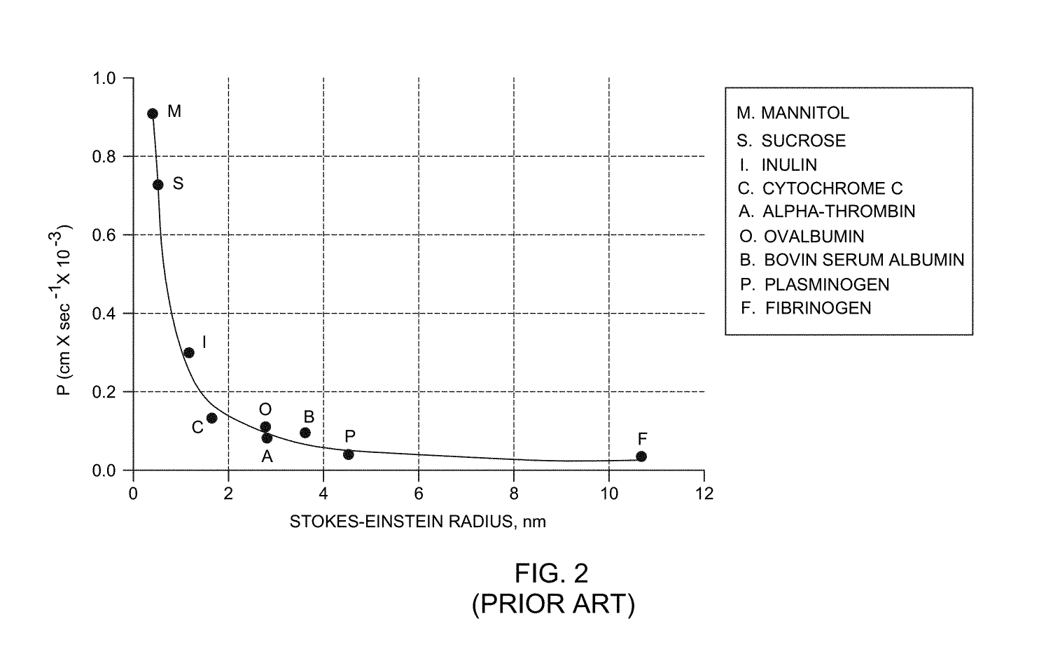

Further, such small molecules may fit between the spaces between the endothelial cells that comprise the capillary walls, which are referred to as inter-endothelial junctions (IEJs). In particular, the IEJs of normal non-neural capillaries permit mass transport of molecules or nanoparticles up to a relatively sharp cutoff at a hydrodynamic diameter of ˜3.5 nm. This is shown in FIG. 2, which depicts a curve illustrating the permeability (P) of endothelial monolayer to molecules of different Stokes-Einstein radii. As shown in FIG. 2, the cutoff size for endothelial permeability (and therefore rapid distribution from blood to interstitial fluid) is approximately 1.5 nm to 2 nm in radius, or 3 nm to 4 nm in diameter. This cutoff, however, may also depend on the form factor, surface charge of the molecule or nanoparticle in question and the potential association of the molecule or nanoparticle with other molecular species that may be present within the body.

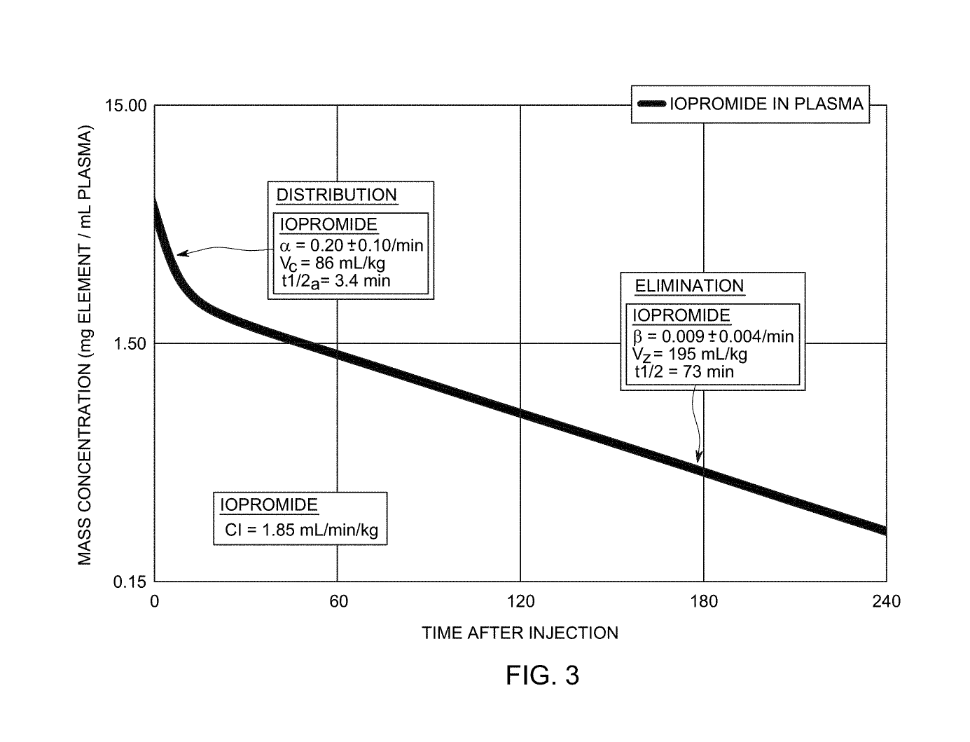

Given this illustration of endothelial permeability, it may be appreciated that, immediately after administration, small-molecule contrast agents begin to distribute from the blood pool into interstitial fluid. Due to this “distribution phase”, the concentration of the contrast agent in the blood pool becomes immediately diluted by a factor of two or more in the first minutes after injection. This is illustrated in FIG. 3 where the concentration of the contrast agent iopromide in pig plasma is illustrated as a function of time, with both a distribution phase and elimination phase being evident.

As shown in FIG. 3, after injection, iopromide, similar to all iodinated small-molecule contrast agents, immediately begins to equilibrate in concentration between the blood (˜6% of body volume) and interstitial fluid (˜21% of body volume). This distribution occurs at a relatively fast rate, with a half-life (T½ₐ) on the order of minutes and is denoted as the distribution phase in FIG. 3. As shown in FIG. 3, the distribution-phase half-life of the iopromide in plasma is much less than 5 minutes. Therefore, the concentration of the molecule in the blood would decrease approximately four fold in much less than 10 minutes due to this initial distribution process alone. However, the drug is simultaneously cleared from the blood by the kidneys at a slower rate, with a T½ on the order of 1-2 hours (denoted as the elimination phase in FIG. 3), resulting in an additional lowering of the concentration in the blood.

Thus, it may be appreciated that certain diagnostic exams, such as venous- and delayed-phase liver CT scans, may be impacted by the reduction in contrast in the imaged volume due to the rapid distribution phase. This results in lower detection rates of certain types of disease, such as venous thrombosis or liver tumors, and poorer delineation of vascular anatomy, than would be obtained if the contrast agent did not distribute and the concentration in the blood pool was therefore higher.

As noted above, the distribution phase is a result of the distribution of the contrast agent from the vasculature to the interstitial fluid. If the size range minimum of the molecules or nanoparticles that comprise the contrast agent can be controlled, the distribution from the blood pool to the interstitial tissue spaces could be mitigated or even eliminated. In this way, the drug can be formulated to reside in the vasculature while the slower elimination phase progresses, during which the drug is eliminated from the body. Since the drug would be largely constrained to the blood volume, or blood pool, within the vasculature and organs until being eliminated, the agent can be designated a “blood pool contrast agent”. More generally, any drug can be designed to have this characteristic, which may be useful in limiting the exposure of certain tissues or organs to the drug.

If the size range maximum of the molecules or nanoparticles that comprise the contrast agent can be controlled, the clearance mechanism can be affected. In this way, the drug can be formulated to clear primarily via the kidneys (i.e., renally). The size limit (e.g., hydrodynamic size limit) for renal clearance is approximately 5.5 nm; however, the renal filtering efficiency depends on several factors including size, shape, and charge.

While rapid distribution and renal clearance are characteristics of small-molecule contrast agents, in comparison, other contemplated agents comprise nanoparticle sizes that are too large to be cleared by the kidneys, i.e., to be cleared renally. For example, nanoparticle-based contrast agents used for preclinical animal imaging generally have sizes in the tens or hundreds of nanometers and are therefore larger than can be efficiently cleared renally. Such agents may be referred to as “blood-pool” or “long-circulating” agents due to their size preventing them from distributing through the inter-endothelial junctions and also preventing renal clearance. Instead, such large particles are cleared through the reticuloendothelial system (RES), resulting in retention in the tissues of the body for an extended time. One disadvantage of the latter is that this retention can interfere with subsequent X-ray/CT exams. In other instances, the retention of the agent in the body may potentially be associated with adverse health consequences in the patient. Therefore, these large-nanoparticle agents are generally less desirable as a general-purpose contrast agent. Alternatively, contrast agents comprising nanoparticles with large sizes have been designed to be biodegradable into small molecules to allow for more rapid elimination, but in this circumstance the elimination time depends on both the rate of biodegradation and rates of distribution and elimination of the biodegraded molecules, leading to complex pharmacokinetic profiles and extended clearance periods.

Although the nanoparticles in some contrast agents are smaller in size than the above-mentioned renal cutoff, these are also smaller in size than the IEJ cutoff, and therefore have PK characteristics like those of small-molecule agents; i.e., they are subject to a rapid distribution phase wherein the agent equilibrates between the blood pool and interstitial fluid of the tissues.

With the preceding discussion of existing contrast agent limitations in mind, it may be appreciated that in the design or construction of an agent, certain properties should be considered. For example, a useful contrast agent should be based on a non-toxic entity that is well tolerated when injected into the bloodstream in large doses (˜10 g-90 g of the dominant X-ray attenuating element), should include an attenuating element(s) that provide good X-ray attenuation in the range of 40-140 keV, should have acceptable viscosity and osmolality, should be adjustable or designable in terms of size and surface chemistry to optimize PK properties, and should have rapid renal clearance. Furthermore, in accordance with certain implementations discussed herein, such a contrast agent may allow customization or selection of particular attenuating element(s) chosen for a specific patient, such as based on patient or anatomy size (e.g., diameter), and would remain in the blood pool rather than distributing from the blood pool into interstitial fluid. With respect to patient size as a consideration, the larger the patient or anatomy being imaged (e.g., the greater the anatomical size), the higher the X-ray energy employed for the imaging operation to obtain sufficient penetration of the patient anatomy for suitable signal-to-noise ratio in reconstructed images. As discussed herein, different contrast materials may be better suited for different X-ray energy ranges.

Another consideration with respect to the design or configuration of a contrast agent as discussed herein is whether the agent is to be employed in a spectral CT or radiographic imaging context, in which projection data is acquired at two or more different X-ray emission spectra (e.g., high- and low-energy in a dual-energy imaging context) or using an energy-discriminating detection mechanism. In such spectral imaging contexts, a suitable attenuating element(s) for a given patient or anatomical size might be different than what might be a suitable attenuating element(s) for conventional single-energy imaging.

With this in mind, the present approach employs a contrast agent (or other administered agent) that is a nanoparticle with a core comprised of element(s) with atomic number (Z) in the range of iodine (Z=53) through bismuth (Z=83); a zwitterionic shell to facilitate acceptable PK, viscosity and osmolality; and a nanoparticle size to facilitate blood-pool distribution and rapid renal clearance, such as a nanoparticle size range of approximately 3.5 nm to 5.5 nm. As discussed herein, however, the specific size range may depend on surface chemistry, particularly surface charge. Therefore, the optimal nanoparticle size may depend to some extent on the specific nanoparticle coating that is used.

The present approach may allow for customization of both the core of the particle (e.g., the payload) and of the shell of the particle, allowing greater flexibility in customizing the properties (e.g., nanoparticle size, surface charge, and form factor) of the overall particle.

With this in mind, with respect to the core of a contrast agent for use in X-ray based imaging, such a core should have suitable X-ray attenuation characteristics for the patient and for the imaging procedure. By way of example, a core or payload material for a contrast agent in accordance with the present approach may be selected from molecules based on elements having an atomic number (Z) including and between approximately 53 (iodine) and 83 (bismuth). Examples of elements in that range that are not known to be toxic and are available at acceptable cost in sufficient commercially-acceptable quantities include iodine, barium, lanthanum, cerium, praseodymium, neodymium, samarium, europium, gadolinium, terbium, dysprosium, holmium, erbium, thulium, ytterbium, lutetium, hafnium, tantalum, tungsten, and bismuth. Likewise, commercially-expensive, or less accessible elements in that range, such as rhenium, osmium, iridium, platinum, and gold, may have limited utility, such as in specialized applications.

With this in mind, selection of the X-ray attenuating core may be based on patient/anatomic considerations, prescribed imaging protocol (e.g., multi- or single-energy), and k-edge attenuation properties of the respective elements.

With respect to patient and/or anatomy size, as noted above, larger patients may be imaged at higher X-ray energies (higher kVp) to obtain sufficient penetration of the greater tissue extent. Attenuation of higher energy X-rays in turn may be improved using higher Z elements as the attenuating material, with certain caveats as discussed below. For example, in terms of selecting a suitable X-ray attenuating core material based on patient size, elements having a Z≤67 may be suitable for smaller patients while elements having a Z≥60 being more suitable for larger patients, with some degree of overlap for elements with Z values between 60 and 67.

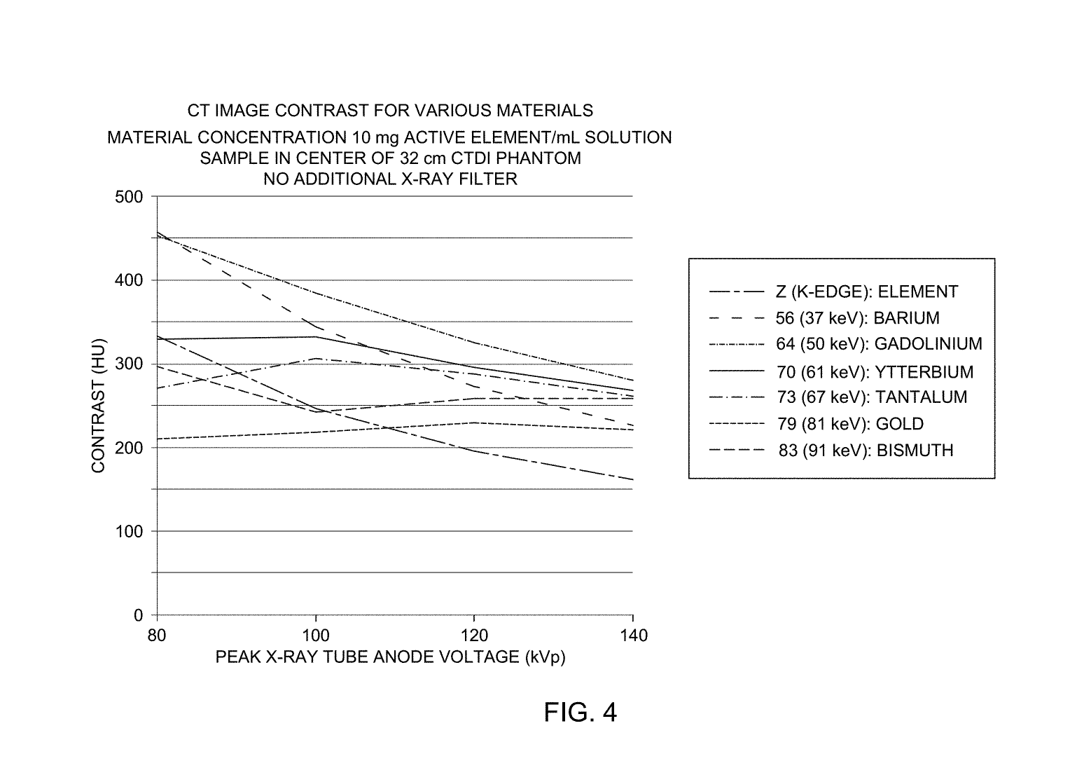

Another consideration in selecting a suitable attenuating element is whether the element exhibits a k-edge effect, which relates to the binding energy of the k-shell electrons. This k-edge effect may manifest as a jump in attenuation over a region of the X-ray emission spectrum. As illustrated in FIG. 4, the contrast of numerous elements suitable for core payloads of an agent as discussed herein are shown over a range of peak X-ray energies. As may be seen in FIG. 4, certain elements of interest exhibit a k-edge effect while others do not. For example, iodine exhibits no k-edge effect at clinically useful X-ray energies (iodine's k-edge energy is at 33 keV, well below typical X-ray energies for whole-body imaging), instead exhibiting monotonically decreasing attenuation with increasing peak voltage in the clinically useful range of X-ray energy. In comparison, those elements for which attenuation does not monotonically decrease or for which attenuation increases at some point in the range of energies exhibit a k-edge effect, such as bismuth at between 100 to 120 keV and tantalum at between 80 to 100 keV.

The significance of such k-edge effect is that it provides another factor to consider in selecting an element for use as an attenuating core of a contrast agent as discussed herein. For example, X-ray tube voltage (kVp) may be selected at least in part based on the patient and/or anatomical size (as discussed above) as well as on the characteristic attenuation of the X-ray attenuating element being used. By way of example, iodine based contrast agents may be unsuitable for use with larger patients due to iodine exhibiting monotonically decreasing attenuation with increasing kVp, and thereby leading to a loss of contrast in larger patients.

Instead, a more suitable attenuating element exhibiting increasing or stable contrast over the energy range of interest may be selected for use with larger patients or anatomical sizes. For single-energy CT, a suitable attenuating element might be one with a k-edge energy slightly below the mean energy of the detected spectrum. For spectral CT (e.g., dual- or multi-energy), a suitable attenuating element might be one with a k-edge positioned within the suitable diagnostic energy range (between 40 keV and 140 keV).

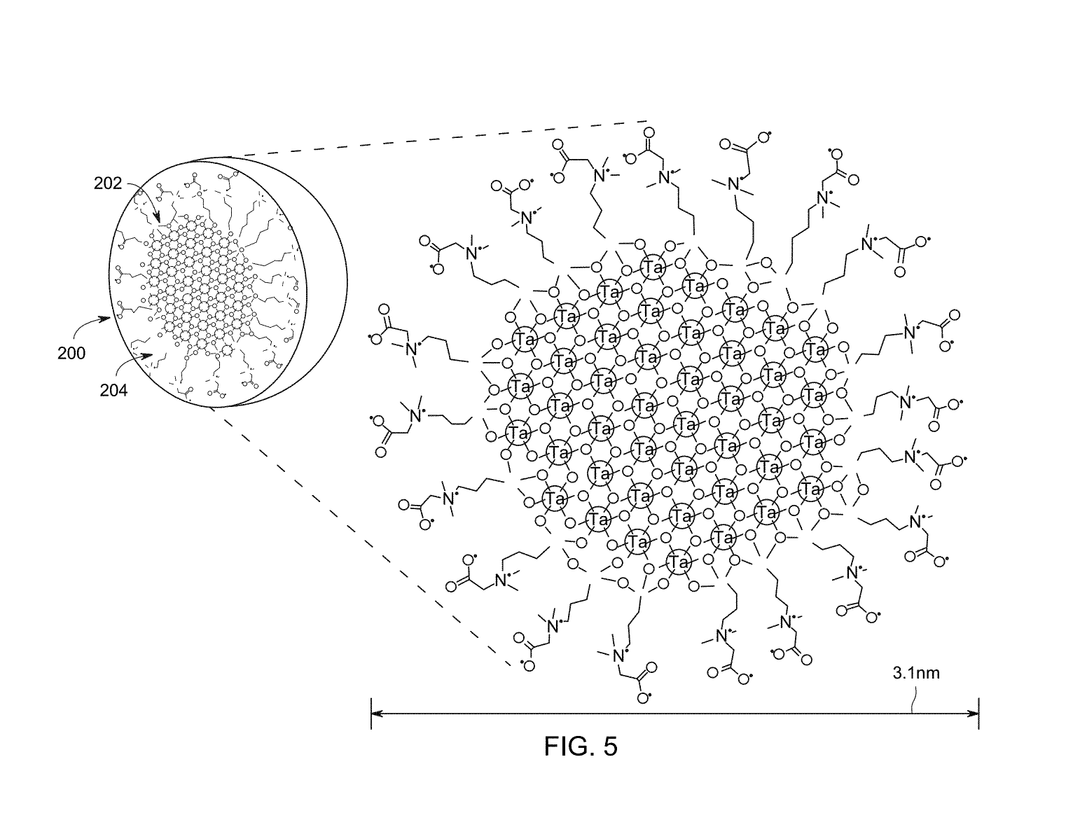

As discussed herein, the selected attenuating element (or other payload) is surrounded by a biocompatible shell. One example of a contemplated contrast agent nanoparticle 200 is shown in FIG. 5, where tantalum oxide core 202 is surrounded by a carboxybetaine zwitterionic shell 204 (TaCZ). In this example, the particle size is polydisperse as assessed by dynamic light scattering, with a nominal size of ˜3.1 nm to 3.5 nm and with a standard deviation of ˜0.5 nm, leading to a size range of ˜2.1 nm to 4.5 nm. As may be appreciated, other suitable biocompatible shells may be employed.

As discussed herein, the present approach allows for some degree of customization in terms of the size of the contrast agent nanoparticle, such as to create nanoparticles large enough to stay in the blood pool (i.e., not distribute into the interstitial fluid, typically corresponding to a size greater than about 3.5 nm), but small enough to renally clear (typically corresponding to a size of less than about 5.5 nm). As noted above, form factor and surface chemistry also may affect these properties, and may therefore also be a factor in determining a suitable size.

Further, the present approach may also be useful in creating contrast agent nanoparticles capable of being used to characterize microvasculature that have larger than normal IEJs or missing IEJs relative to healthy vasculature, such as occurs in tumors or inflamed tissues. In particular, contrast agent (or treatment) particles having a size capable of mass transport through the tumor IEJs but not the IEJs of healthy vasculature may be useful in detecting tumors and inflammation, characterizing the tumor microvasculature, and/or allowing early evaluation of response to therapy. Thus, such agents would remain in the blood pool except within tumor or inflamed tissue. Conversely, in some normal tissues such as the liver, endothelial sinusoids are highly porous to larger-sized nanoparticles due to the presence of endothelial fenestrations. In such porous tissues, the reduction or loss of porosity is a signal of disease, such as in liver fibrosis. Thus, agents with a nanoparticle size that allows faster mass transport through healthy sinusoid endothelial fenestrations but reduced mass transport through diseased sinusoid endothelial fenestrations would be useful to detect and monitor disease in such tissues.

By separating the agent into two discrete aspects, i.e., a payload or core aspect and a shell aspect, two benefits are realized: (1) the functions of attenuation and biocompatibility are separately provided by the core and the shell, respectively, and therefore the design of either function can be varied somewhat independently of the other; (2) the size of the nanoparticle can be tailored to achieve optimal pharmacokinetics (PK) (as discussed above), without affecting the functions of attenuation or biocompatibility (with the caveat that that particle size may affect viscosity and osmolality). This may allow a high degree of customization both with respect to patient and imaging procedure. For example, a contrast agent may be generated that provides both contrast enhancement during the early (arterial) phase of a CT exam equal to or higher than that provided by conventional iodinated agents (with agents injected at equal mass concentration) due to strategic selection of the attenuating material, and the contrast during the later (venous and delayed) phases can be substantially higher using a size-optimized agent than with conventional small-molecule agents (which distribute into the interstitial fluid).

With the preceding in mind, CT scans of rats and swine have been performed using the TaCZ contrast agent shown in FIG. 5. In these studies, an iodinated small-molecule agent was compared with the prototype TaCZ agent that, as noted above, is a nanoparticle with a tantalum oxide core and a carboxybetaine zwitterion shell. As noted above, the particle size of this realization of TaCZ is polydisperse, with a nominal size of ˜3.1 to 3.5 nm and with a standard deviation of ˜0.5 nm, leading to a size range of ˜2.1 nm to 4.5 nm and thus including some particles smaller than the desired 3.5 nm threshold, i.e., the IEJ cutoff.

Results were obtained in two forms: clinical benefit via image quality assessment and pharmacokinetic (PK) modeling via blood samples.

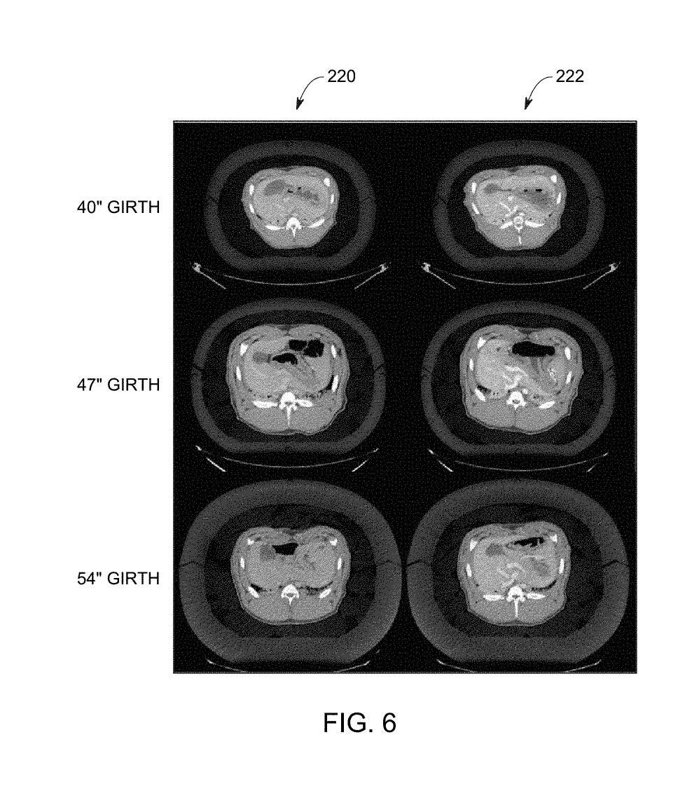

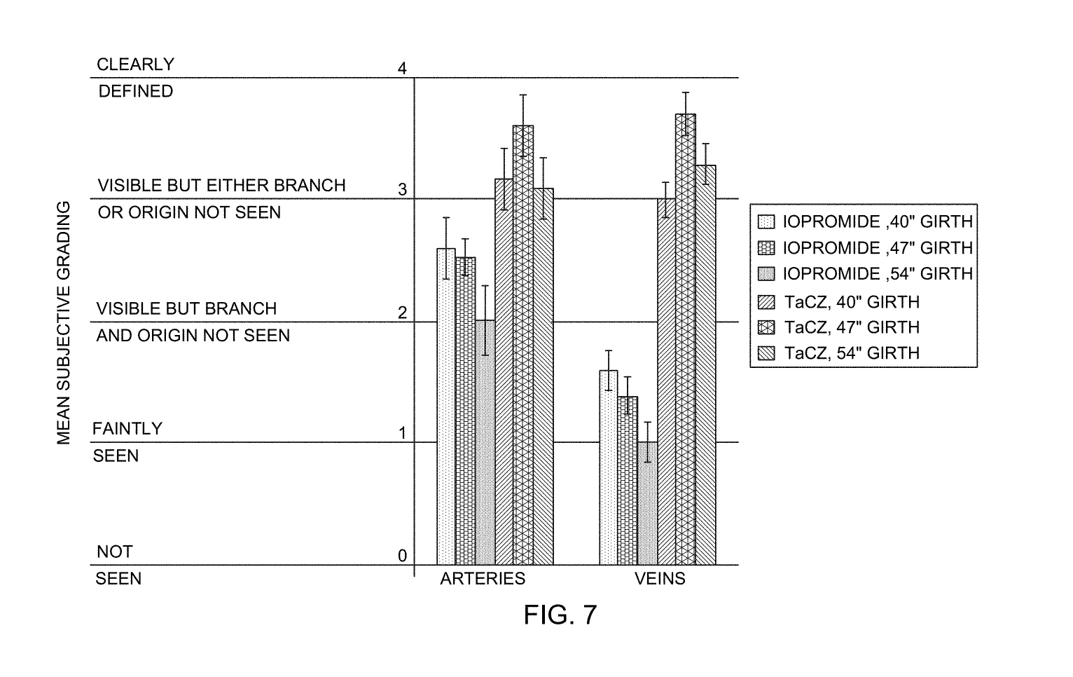

The clinical benefit was observed by comparing CT scans of swine, during which the same animals were scanned sequentially using either an iodinated small-molecule clinical contrast agent or TaCZ. The scans were performed one day to one week apart, and the scan sequence was randomized. During scanning, the pigs were encased in plastic fat-equivalent encasements to emulate a range of large patient sizes. Scans were performed in the pigs' livers at several time points from 30 to 300 seconds after injection. Image quality at each time point was graded by radiologists using predefined criteria such as image contrast in specified vessels. The results are illustrated in FIGS. 6 and 7.

In FIG. 6, images 220 on the left were acquired using a conventional iodinated contrast agent, iopromide, while images 220 on the right were acquired using the TaCZ nanoparticle contrast agent described above. Vertically, the images are arranged based on patient size. As shown in FIG. 6, as patient size increases (and X-ray energy correspondingly increases) the image contrast enhancement provided by the iodine-based agent decreases relative to the contrast enhancement provided by the TaCZ.

In FIG. 7, the results of the multi-reader assessment are provided in graphical form. In addition to the effect of the active element, tantalum, the pharmacokinetics of the contrast agent influences the image contrast, especially in the images of the veins, which are enhanced at later times, after the blood has passed through the capillaries and the small-molecule contrast agent has begun to distribute to interstitial fluid. Conversely, the concentration of the larger TaCZ particle has not decreased substantially in the intervening time.

Thus, these results demonstrate the benefit of the use of contrast agents based on higher-Z elements than iodine in large patients, such as in the core of a composite contrast agent as discussed herein. In addition, the benefit of the size of the particle being tailored to remain within the blood pool is also demonstrated in the venous phase scans assessed in FIG. 7, where the blood-pool distribution of TaCZ yields a far higher image contrast, and therefore vessel detectability, than the small-molecule agent.

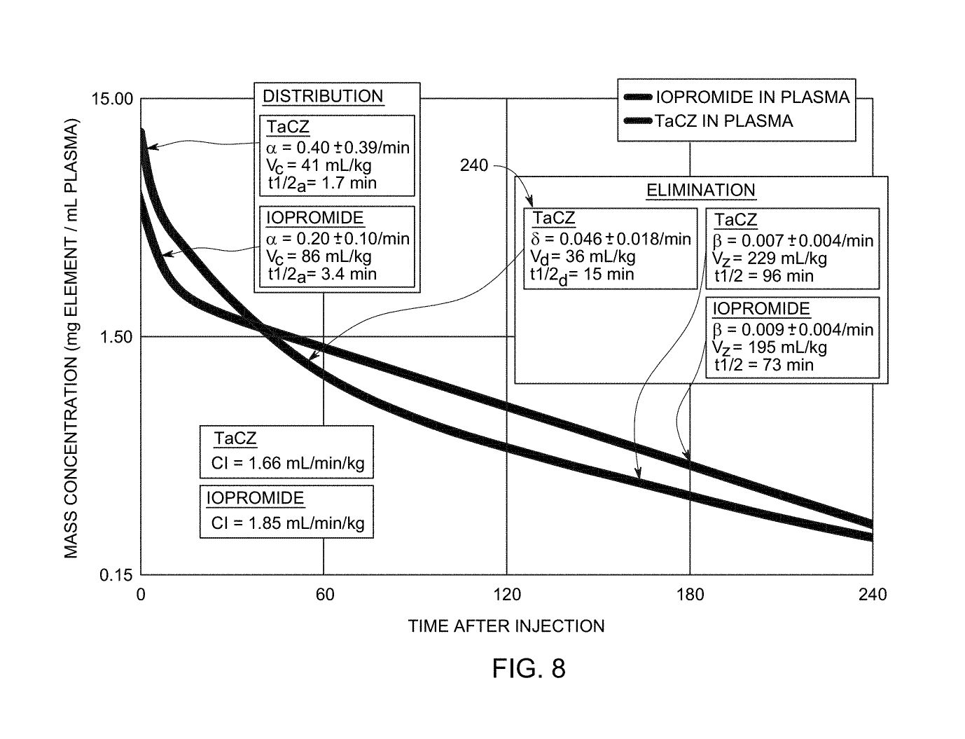

In a separate analysis, PK modeling was obtained by analyzing the concentration of the active element (iodine or tantalum) in blood samples taken from 2 to 240 minutes after contrast agent injection. The results show two distinct exponential components that based on their rate constants can be assigned to the distribution and elimination processes noted above, as shown in FIG. 8.

However, as seen in FIG. 8, when using TaCZ, three distinct exponential processes were observed. In particular, like iodinated small molecules (e.g., the iopromide), some fraction of the nanoparticle agent TaCZ distributes to interstitial fluid with T½ₐ on the order of minutes, and T½ on the order of hours. In this figure, an average (n=6 pigs) distribution T½ₐ=1.7 minutes is shown and an average clearance T½=96 minutes. However, the TaCZ curve contains an exponential component not found in the iopromide curve, which has a T½_{d}=15 minutes. This may be attributed to the portion of the size distribution of the injected agent that includes nanoparticles larger than the IEJ cutoff size. Therefore, the concentration of the tantalum in the blood (from the larger nanoparticles) is not diluted as much as an iodinated agent, resulting in a tantalum concentration at 1 to 3 minutes that is twice as high as iodine's. This leads to higher image contrast using TaCZ versus iopromide at the clinically-important imaging times. Furthermore, it appears that these large nanoparticles are cleared from the blood by the kidneys at T½_{d}=15 minutes, resulting in faster clearance than iopromide.

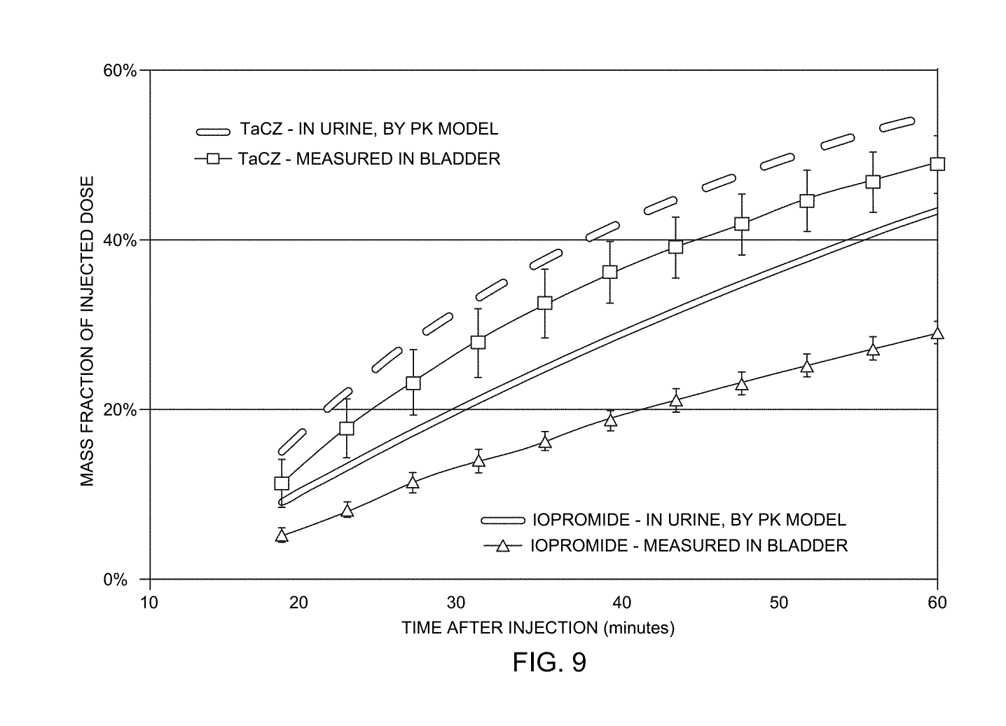

It may be noted that the TaCZ curve of FIG. 8 shows that the concentration of tantalum in the blood is decreasing, but it does not show whether this is due to distribution or to clearance. Therefore, the tantalum and iodine dose that was excreted into the urinary bladders of the pigs was measured at several time points to test the hypothesis that the exponential with T½_{d}=15 minutes corresponds to renal clearance and not to a slow distribution process. To generate these results, the amount of the injected agent that would be expected to accumulate in the urine was estimated assuming the hypothesis was correct. The amount of injected agent was then measured in the bladder. Given that the model includes all urine (including that in the kidneys, ureters, and bladder) but that only urine in the bladder was measured, the results generally agreed (FIG. 9), thus supporting the hypothesis.

Note that this concept can be extended to include other drug- or agent-delivery applications that benefit from payload interchangeability, blood-pool distribution, and renal clearance. These include contrast agents for PET, MRI and other imaging modalities. Other uses of the approaches discussed herein include, but are not limited to, the delivery of cancer treatment drugs (such as where the nanoparticles leak from tumors' permeable microvessels and the nanoparticle shell is designed to be digested by the tumors), the delivery of radioactive materials as the payload (such as where a nanoparticle shell of the particles discussed herein is functionalized to attach to pathologies such as tumors, and the drug/payload can serve as a PET tracer, but with the advantage of having the blood-pool distribution as provided by the particle's size and coating characteristics), and the delivery of multiple or mixed payloads within a shared nanoparticle shell, including multiple X-ray attenuating element(s) having differing attenuating properties, and/or radioactive payload(s), and/or therapeutic drug(s).

In addition, an injection or administration to a patient can comprise a mixture of multiple or distinct particle types, each with the same or different PK characteristics and/or with different payloads. For example, the different payloads can be X-ray attenuating element(s), radioactive payload(s), and/or therapeutic drug(s). For example, using a multiple-syringe injector, these agents can be injected simultaneously or sequentially. For instance, one specific application of using separately-timed injections would be to inject contrast agents with different attenuating elements at different times. Such an approach would allow using spectral imaging to simultaneously image veins and liver parenchyma (using material decomposition to highlight the earlier injection) and arteries (using material decomposition to highlight the later injection), thus reducing X-ray dose and improving workflow.

In general, the present approaches may be desirable in any agent delivery context where controlled pharmacokinetic distribution and/or renal clearance are factors.

Technical effects of the invention include a nanoparticle with a size large enough to remain in the blood pool but small enough to be renally cleared. Such a particle has important benefits over a smaller entity (a molecule or nanoparticle similar in size to current small-molecule contrast agents). For example, the agent will have a higher plasma concentration and produce higher image contrast than a smaller entity in contrast imaging contexts; a larger particle will have a substantially higher per-particle payload than a smaller particle because the volume and therefore mass of the core increases as the cube of its radius; therefore, fewer particles are required for a given concentration in contrast imaging or treatment contexts; osmolality and viscosity are lower if fewer larger particles are used to provide a given concentration; and the renal clearance rate is higher.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An agent that can be injected into a subject, wherein the agent comprises:
nanoparticles or molecules sized to effect a specific degree of distribution or lack of distribution between tissues, organs, or bodily compartments of the subject while still being eliminated by the kidneys.

2. The agents of claim 1, wherein the agent comprises nanoparticles comprising:
a core; and
a shell surrounding the core.

3. The agent of claim 1, wherein the nanoparticles or molecules contain one or more of X-ray attenuating elements,

4. The agent of claim 3, wherein the one or more X-ray attenuating elements comprise elements that have an atomic number from 53 to 83.

5. The agent of claim 1, wherein the agent is injected into the veins or arteries and the agent comprises nanoparticles or molecules that have a size range maximum selected to be smaller than about 3.5 nm so as to distribute from the blood pool during imaging.

6. The agent of claim 1, wherein the agent is injected into the veins or arteries and the agent comprises nanoparticles or molecules that have a size range minimum larger than about 3.5 nm and a size range maximum smaller than about 5.5 nm so as to remain in the blood pool during imaging.

7. The agent of claim 2, wherein the core comprises one or more elements or molecules having different X-ray attenuation properties, gamma ray emission properties, magnetic properties, or therapeutic properties.

8. A method of performing a contrast-enhanced image acquisition, comprising:
determining a size of a patient or an anatomical region within the patient to be imaged;
based on the size of the patient or anatomical region, determining an X-ray energy spectrum to be used to acquire one or more images of the patient or anatomical region within the patient;
based on one or both of the anatomical size or the X-ray energy spectrum, selecting one or more X-ray attenuating elements to be used as a constituent of a contrast agent;
administering the contrast agent to the patient, wherein the contrast agent comprises nanoparticles or molecules having a size selected so as to effect a specific degree of distribution or lack of distribution between tissues, organs, or bodily compartments of the patient while still being eliminated by the kidneys; and
acquiring one or more contrast-enhanced images of the patient,

9. The method of claim 8, wherein the contrast agent is injected into the veins or arteries and the contrast agent comprises nanoparticles or molecules that have a size range maximum selected to be smaller than 3.5 nm so as to distribute from the blood pool during imaging.

10. The method of claim 8, wherein the contrast agent is injected into the veins or arteries and the contrast agent comprises nanoparticles or molecules that have a size range minimum larger than about 3.5 nm and a size range maximum smaller than about 5.5 nm so as to remain in the blood pool during imaging.

11. The method of claim 8, wherein the one or more X-ray attenuating elements comprise elements that, have an atomic number from 53 to 83.

12. The method of claim 8, wherein the contrast agent comprises a nanoparticle comprising:
a core containing one or more of the X-ray attenuating elements; and
a shell surrounding the core.

13. The method of claim 12, wherein the shell comprises a zwitterionic shell.

14. The method of claim 8, wherein the one or more X-ray attenuating elements are selected based on whether the one or more X-ray attenuating elements have a k-edge energy within an X-ray energy range of interest.

15. The method of claim 8, wherein determining the X-ray energy spectrum based on the size of the patient or anatomical region comprises selecting a higher-energy X-ray spectrum for larger sizes of the patient or anatomical region.

16. A method for performing a procedure using one or more types of drugs that can be injected into a patient, the method comprising:
administering the one or more types of drugs to a patient as part of a procedure, wherein the drugs, when more than one is present, may be injected simultaneously or sequentially, and wherein one or more of the one or more types of drugs comprise nanoparticles or molecules having a size selected so as to effect a specific degree of distribution or lack of distribution between tissues, organs, or bodily compartments of the patient while still being eliminated by the kidneys.

17. The method of claim 16, wherein at least one of the one or more drugs contains one or more X-ray attenuating elements.

18. The method of claim 16, wherein at least one of the one or more drugs contains one or more magnetic elements, therapeutic drugs, gamma-ray emitting elements; or molecules comprising one or more elements with one or more of these properties.

19. The method of claim 17, wherein the one or more X-ray attenuating elements are selected based on whether the one or more X-ray attenuating elements have a k-edge energy within an X-ray energy range of interest.

20. The method of claim 17, wherein one of the one or more drugs that contain one or more X-ray attenuating elements have X-ray attenuating properties that differ from the X-ray attenuating properties of one or more of the other types of drugs.

21. The method of claim 17, wherein the X-ray attenuating properties of the one or more types of drugs that contain one or more X-ray attenuating elements are selected for specific imaging or therapeutic treatment conditions or objectives.

22. The method of claim 16, wherein at least one of the one or more types of drugs is injected into the veins or arteries and the drug comprises nanoparticles or molecules that have a size range maximum smaller than about 3.5 nm so as to distribute from the blood pool during imaging.

23. The method of claim 16, wherein at least one of the one or more types of drugs is injected into the veins or arteries and the drug comprises nanoparticles or molecules that have a size range minimum larger than about 3.5 nm and a size range maximum smaller than about 5.5 nm so as to remain in the blood pool during imaging.

24. The method of claim 16, wherein at least one of imaging properties and pharmacokinetic properties of at least one of the one or more types of drugs are selected for at least one of imaging or therapeutic treatment conditions or objectives.

25. The method of claim 16, wherein at least one of the one or more types of drugs comprises a nanoparticle comprising:
a core containing; and
a shell surrounding the core,
wherein at least one of the core and shell differ for different types of nanoparticles.

26. The method of claim 25, wherein the shell comprises a zwitterionic shell.
